(19) United States
(12) Patent Application Publication
(10) Pub. No.: US 2022/0204466 A1
(43) Pub. Date: Jun. 30, 2022

(54) PROCESS FOR MANUFACTURING HYDROXYMETHYLFURFURAL

(71) Applicant: PURAC BIOCHEM B.V., Gorinchem (NL)

(72) Inventors: Maria Joao DAMASO RODRIQUES BRINQUETE PROENCA, Gorinchem (NL); Cornelis Johannes Govardus VAN STRIEN, Gorinchem (NL)

(73) Assignee: PURAC BIOCHEM B.V., Gorinchem (NL)

(21) Appl. No.: 17/609,670

(22) PCT Filed: Jun. 4, 2020
(86) PCT No.: PCT/EP2020/065511

§ 371 Date: Nov. 8, 2021

(30) Foreign Application Priority Data
Jun. 6, 2019 (EP) 19178669.8

(51) Int. Cl. C07D 307/50

(52) U.S. Cl. CPC C07D 307/50

(57) ABSTRACT

A process for producing 5-hydroxymethylfurfural (HMF) including a) a step of converting a carbohydrate into HMF, the converting step including providing a reaction medium including carbohydrate, catalyst, water, tetrahydrofuran (THF), and salt to form a biphasic solvent system including an aqueous phase and a THF phase and b) a step of separating the THF phase and the aqueous phase, to provide a separate THF phase and a separate aqueous phase, wherein an organic quaternary ammonium salt is present. The process results in a high conversion of carbohydrate and in the formation of HMF in high selectivity with low formation of side products, together with an efficient extraction of HMF into the THF phase.

The present invention pertains to a process for manufacturing 5-hydroxymethylfurfural (HMF) from carbohydrates.

Hydroxymethylfurfural (HMF) is readily accessible from renewable resources like carbohydrates and is a suitable starting source for the formation of various furan monomers which are used for the preparation of non-petroleum-derived polymeric materials. HMF has the following formula:

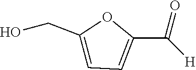

Methods for manufacturing HMF have been described in the art.

U.S. Pat. No. 7,572,925 describes a process for manufacturing HMF from carbohydrates which comprises dehydrating a carbohydrate feedstock solution, optionally in the presence of an acid catalyst, in a reaction vessel containing a biphasic reaction medium comprising an aqueous reaction solution and a substantially immiscible organic extraction solution, resulting in the formation of a biphasic system wherein HMF is present in the organic extraction solution. The organic extraction solution comprising HMF is then separated from the aqueous layer which comprises the catalyst and many of the side products, and the HMF is recovered from the organic extractant solution. Extractants used in this reference include alcohols, with 1-butanol being preferred, ketones, with methyl-isobutylketone being preferred, and chlorinated alkanes, with dichloromethane being preferred.

Y. Román-Leshkov and J. A. Dumesic (Top Catal (2009) 52:297-303) describe the use of THF as extractant solvent in biphasic systems, with NaCl being used to ensure the formation of a biphasic system, since THF and water are miscible.

It has been found, however, that there is need in the art for a process which combines a high conversion of carbohydrate sources into HMF and a high extraction efficiency of the HMF with a high selectivity for HMF, and low formation of other compounds. The present invention provides such a process.

The invention pertains to a process for producing 5-hydroxymethylfurfural (HMF) comprising

a) a step of converting a carbohydrate into HMF, the converting step comprising providing a reaction medium comprising carbohydrate, catalyst, water, tetrahydrofuran (THF), and salt to form a biphasic solvent system comprising an aqueous phase and a THF phase and

b) a step of separating the THF phase and the aqueous phase, to provide a separate THF phase and a separate aqueous phase, characterised in that an organic quaternary ammonium salt is present.

It has been found that the specific combination of THF as solvent and the presence of an organic quaternary ammonium salt results in a process with surprising and attractive properties. It was found that the process according to the invention results in a high conversion of carbohydrate and in the formation of HMF in high selectivity with low formation of side products, together with an efficient extraction of HMF into the THF phase. Further advantages of the present invention and specific embodiments will become apparent from the further specification.

It is noted that Q. Cao et al., Appl. Cat. A General (pp 98-103), 2011, describes the use of tetraethyl ammonium chloride (TEAC) and other ammonium salts in the transformation of fructose into HMF, either in as such, or in the presence of tetrahydrofuran (THF). The fructose and salt are combined to form a melt.

This reference is not directed to biphasic extraction using a water/extractant system.

WO 2016/059205 relates to a process for the production and isolation of HMF from saccharides. CN 101906088 relates to a method for preparing HMF, in particular to use an eutectic mixture of ammonium salt and sugar to efficiently convert sugar into HMF. WO 2014/180979 relates to a process for dehydration of monosaccharides having 6 carbon atoms (hexoses), disaccharides, oligosaccharides and polysaccharides deriving therefrom to yield HMF. It is said the HMF is obtained in high yield and high purity. These references are also not directed to biphasic extraction using a water/extractant system.

The invention will be discussed further below.

The starting material in the process according to the invention is a carbohydrate. Carbohydrates are produced by photosynthetic plants and contain only carbon, hydrogen, and oxygen atoms. Examples of carbohydrates include lignin, sugars, starches, celluloses, and gums. Compound particularly suitable for use in the present invention include in particular C5 sugars such as arabinose, xylose and ribose; C6 sugars such as glucose, fructose, galactose, rhamnose and mannose; and C12 sugars such as sucrose, maltose and isomaltose. Glucose, fructose, and sucrose have been found to be particularly suitable. Glucose and sucrose may be preferred in view of their high availability. The use of sucrose may be particularly attractive because it is generally available in the solid form.

An organic quaternary ammonium salt is used in the present invention.

The term organic quaternary ammonium salt is intended to refer to a salt consisting of an organic quaternary ammonium cation and an anion.

The organic quaternary ammonium cation is of the formula R₁R₂R₃R₄N⁺, wherein at least one of R₁, R₂, R₃, and R₄ is a C1-C20 hydrocarbon group. The others of R₁, R₂, R₃, and R₄ may be independently selected from C1-C20 hydrocarbon groups and hydrogen. In the context of the present specification the term organic quaternary ammonium thus also covers compounds wherein one, two, or three of R₁, R₂, R₃, and R₄ are hydrogen.

It may be preferred for at least two of R₁, R₂, R₃, and R₄ to be a C1-C20 hydrocarbon group, in particular at least three, more in particular at least four. The C1-C20 hydrocarbon groups may be the same or different.

The term C1-C20 hydrocarbon group encompasses alkylgroups, arylgroups, arylalkyl groups and alkylaryl groups. The C1-C20 hydrocarbon groups may be straight-chain or branched, and may or may not be substituted with one or more substituent groups selected from OH, NH2, SH, COOH, SO3, and PO4.

It may be preferred for the hydrocarbon group discussed above to be a C1-C10 hydrocarbon group, in particular a C1-C5 alkylgroup or a C6-C8 alkylaryl or arylalkylgroup. Examples of some preferred hydrocarbon groups are methyl, ethyl, hydroxyethyl, propyl, benzyl, and phenyl.

The anion in the organic quaternary ammonium salt is not critical, as long as the salt has a high solubility in water. Examples of suitable anions are halides including fluoride, chloride, bromide, and iodide, with chloride being preferred for reasons of availability. Other suitable inorganic anions include nitrate, sulphate and phosphate.

Organic anions may also be used, e.g., carboxylate anions.

Examples of preferred organic quaternary ammonium salts include tetramethylammonium chloride, tetraethylammonium chloride, benzyltrimethylammonium chloride, benzyltriethylammonium chloride, phenyltrimethylammonium chloride, phenyltriethylammonium chloride, 1-hydroxyethyltrimethylammoniumchloride (cholinechloride), tetramethylammonium bromide, tetraethylammonium bromide, benzyltrimethylammonium bromide, benzyltriethylammonium bromide, phenyltrimethylammonium bromide, and phenyltriethylammonium bromide. The use of chloride compounds is considered preferred. The use of tetramethylammonium chloride, tetraethylammonium chloride, and 1-hydroxyethyltrimethylammoniumchloride (cholinechloride), is considered more preferred, with 1-hydroxyethyltrimethylammoniumchloride (cholinechloride) being preferred in particular.

It is a key feature of the organic quaternary ammonium salt that it has a high solubility in water. This is required to ensure the formation of a biphasic system. In general, the organic quaternary ammonium salt has a solubility in water of at least 10 wt. %, in particular at least 40 wt. %, at the desired operating temperature.

The catalyst used in the present invention may be any catalyst known in the art for the conversion of carbohydrates to HMF.

Examples of suitable catalysts are metal salts such as metal halides, in particular metal chlorides. Examples of suitable metals are Cr, Mo, W, Fe, Ni, Co, Cu, Sn, and Al. Inorganic acid such as HCl, H2SO4, H3PO4, H3BO3, may also be used. Solid acid catalyst such as molecular sieves, silica-alumina, and ion exchange resins may also be used.

The use of halides of chromium and aluminium, in particular chromium chloride and aluminium chloride is considered preferred. The use of molecular sieves, in particular zeolites, is also considered preferred.

In the process according to the invention a reaction a reaction medium is provided comprising carbohydrate, catalyst, water, tetrahydrofuran (THF), and organic quaternary ammonium salt. Of these, the carbohydrate and quaternary ammonium salt will primarily be present in the aqueous phase. Therefore, the amounts of these components will be expressed calculated on an aqueous medium comprising these compounds. It is noted that the compounds can be added to the system in any sequence, and thus not necessarily through the aqueous medium.

The carbohydrate is generally present in an amount of 1-40 wt. %, calculated on the total of carbohydrate, water, and salt. A value below 1 wt. % will make the process economically less attractive. A value above 40 wt. % may lead to processing difficulties. It may be preferred for the amount of carbohydrate to be in the range of 5-30 wt. %, in particular 5-20 wt. %. The organic quaternary ammonium salt will be present in an amount which is sufficient to ensure the formation of a biphasic system. It will generally be present in an amount of at least 10 wt. %, calculated on the total of carbohydrate, water, and salt, in particular in an amount of at least 20 wt. %, more in particular in an amount of at least 30 wt. %. The upper limit of the amount of organic quaternary ammonium salt will be determined by the solubility of the salt in the reaction mixture. The presence of insoluble salts is to be avoided. In general, a maximum of 80 wt. %, calculated on the total of carbohydrate, water, and salt may be mentioned.

The water may be present in an amount of at least 5 wt. %, preferably in an amount of at least 10 wt. %, more preferably in an amount of at least 15 wt. %, calculated on the total of carbohydrate, water, and salt. In some embodiments, the water is present in an amount of at most 95 wt. %, calculated on the total of carbohydrate, water, and salt, preferably in an amount of at most 90 wt. %, more preferably in an amount of at most 85 wt. %. It may be preferred to the amount of water to be in the range of 20-80 wt. %, calculated on the total of carbohydrate, water, and salt.

In addition to the organic quaternary ammonium salt, it is possible that soluble inorganic salts are present during the reaction. In general, if present, the soluble inorganic salt will be selected from inorganic soluble salts of alkaline earth metals and alkali metals, e.g., from soluble salts of Na, K, Mg, and Ca, e.g., chloride salts, (soluble) sulphate salts, and nitrate salts. Examples are NaCl, MgCl2, CaCl₂), KCl, Na₂SO₄, K₂SO₄, and NaNO₃.

If present, the inorganic salt will generally be used in an amount of at most 30 wt. % of the organic quaternary ammonium salt, in particular at most 20 wt. %, more in particular at most 10 wt. %.

Where the catalyst is a homogeneous catalyst, it is generally used in an amount of 0.3-10 wt. %, calculated on the amount of the carbohydrate. Where the catalyst is a heterogeneous catalyst, i.e., a solid catalyst such as a molecular sieve or an ion exchange resin, it is not possible to give a general range for the amount of catalyst. It is within the scope of the skilled person to select a suitable amount of heterogeneous catalyst.

The amount of THF present in the reaction mixture may vary within wide ranges, also depending on how the process is carried out. In general, the use of larger amounts of THF will lead to an increased amount of extracted HMF. On the other hand, where the reaction mixture contains a very large amount of THF, substantially more than required to extract the HMF generated, the equipment and utility costs will increase without extra benefit being obtained.

In one embodiment, where the process is carried out in batch mode, it may be preferred for the weight ratio of THF to aqueous solution containing carbohydrate and salt to be in the range of 0.05:1 to 10:1, preferably 0.2:1 to 5:1, in particular in the range of 1:1 to 2:1. In one embodiment, where the process is carried out in continuous mode, it may be preferred for the weight ratio of THF to aqueous solution containing carbohydrate and salt to be in the range of 0.05:1 to 10:1, preferably 0.2:1 to 3:1, in particular in the range of 0.5:1 to 2:1. The latter ratio may be preferred, as it allows for a more facile extraction process.

Reaction temperature is generally in the range of 80-180° C., in particular in the range of 90-160° C., more in particular in the range of 100-150° C.

The reaction time will depend on the ration temperature, with lower reaction temperatures generally causing longer reaction times. In general, the reaction time may be in the range of 1 minute to 4 hours, preferably 5 minutes to 2 hours.

The process results in the formation of a biphasic system comprising an aqueous phase and a THF phase.

Of the compounds in the reaction mixture, the quaternary ammonium salt will be primarily present in the aqueous phase. In other words, of the total amount of quaternary ammonium salt, at least 90% will be present in the aqueous phase, more in particular at least 95%, still more in particular at least 98%.

Where the catalyst is a homogeneous catalyst it will also primarily be present in the aqueous phase. In other words, of the total amount of homogeneous catalyst, at least 90% will be present in the aqueous phase, more in particular at least 95%, still more in particular at least 98%. As will be evident to the skilled person, heterogeneous catalysts are not present in the liquid phase.

The amount of HMF present in the THF phase and in the aqueous phase will depend on the amount of THF present. In a batch process it may be preferred if at least 20% of the HMF in the reaction mixture is present in the THF phase, preferably at least 40%, and in particular at least 60%, more in particular at least 80%, still more in particular at least 90%. In a continuous process it may be preferred if at least 10% of the HMF in the reaction mixture is present in the THF phase, preferably at least 20%, and in particular at least 40%, more in particular at least 50%.

Unconverted carbohydrate will primarily be present in the aqueous phase. In other words, of the total amount of unconverted carbohydrate, at least 80% will be present in the aqueous phase, more in particular at least 90%, still more in particular at least 95%.

The biphasic system comprising an aqueous phase and a THF phase is subjected to a phase separation step, resulting in the formation of a separate THF phase and a separate aqueous phase. Separating the aqueous phase from the THF phase can be done by methods known in the art for separating a liquid-liquid two-phase system. Examples of suitable apparatus and methods for liquid-liquid separation include decantation, settling, centrifugation, use of plate separators, use of coalescers, and use of hydrocyclones. Combination of different methods and apparatus may also be used.

In one embodiment, the separation step is carried out at a temperature which is at or below the temperature of the reaction step, as a lower temperature may improve the distribution coefficient. The temperature during the separation step may, e.g., be in the range of 20-180° C., in particular 20-130° C., more in particular 20-100° C.

The separated THF phase which contains HMF may be processed as desired. In one embodiment, THF is removed by evaporation from the mixture of THF and HMF. If so desired, water may be added to the THF phase which contains HMF to avoid the azeotrope between water and HMF. As THF and water are fully miscible, this will result in the formation of a monophasic mixture of HMF, THF, and water, from which THF can be removed through evaporation, resulting in the formation of a solution of HMF in water.

If so desired, THF can be recycled to the reaction step, optionally after purification. For example, side products such as formic acid and other components can be removed by partial condensation.

The aqueous phase comprises organic quaternary ammonium salt and, where the catalyst is homogeneous, catalyst. The aqueous phase may also comprise unconverted carbohydrate. If so desired, the aqueous phase can be recycled to the reaction step.

The aqueous phase may contain solid contaminants formed during the reaction, often indicated as hum ins. They can be removed by solid-liquid separation in manners known in the art, e.g., filtration, centrifugation, settling, and combinations thereof. Additionally, the aqueous phase may be concentrated by removal of water, e.g., through evaporation, to compensate for water formed during the reaction and, in some cases, water added when the carbohydrate is added in the form of a syrup.

The HMF may be processed as desired.

In one embodiment, HMF is converted to furan-dicarboxylic acid (FDCA). FDCA may in turn, be reacted with ethyleneglycol in a polycondensation reaction to form poly(ethylenefurandicarboxylate) (PEF).

Conversion of HMF to FDCA is known in the art. It can, e.g., take place through fermentative biooxidation, e.g., as described in WO2011/026913.

Formation of PEF from FDCA and polyethylene glycol through polycondensation is also well known in the art. It is, e.g., described in EP3116932, EP3116933, and EP3116934. Neither process requires eilucidation here.

The present invention also pertains to the use of the HMF obtained by the process according to the invention in the manufacture of FDCA through fermentative biooxidation, and to the use of the FDCA thus obtained in the manufacture of PEF through polycondesntaion of the H DCA with ethylene glycol.

Combinations of various embodiments of the process according to the invention may be combined, unless they are mutually exclusive.

The invention will be elucidated by the following examples, without being limited thereto or thereby.

### EXAMPLE 1: GLUCOSE TO HMF

In an experiment according to the invention, an aqueous solution of 10 wt. % of glucose, 5 mole % of CrCl3 as catalyst (calculated on glucose) and 63 wt. % choline chloride was combined with THF in a weight/weight ratio of 1:1, forming a biphasic mixture, and brought to a reaction temperature of 130° C.

The results are presented in table 1 below:

TABLE 1
Results

| | | | | Selectivity towards |
|---|---|---|---|---|
| | | HMF | HMF | HMF [mol HMF |
| | Glucose conversion [mol | concentration - | concentration- | produced/mol |
| | glucose reacted/mol | organic phase | aqueous phase | glucose converted, |
| t [min] | glucose fed, %] | [wt %] | [wt %] | %] |
| 10 | 11.0 | 0.02 | 0.01 | 3.8 |
| 20 | 44.0 | 0.91 | 0.36 | 40.6 |
| 30 | 69.2 | 2.00 | 0.81 | 57.0 |

As can be seen from Table 1, the process according to the invention makes it possible to produce HMF from glucose with good conversion and good selectivity.

### EXAMPLE 2: COMPARISON OF CHOLINE CHLORIDE OR NACL IN THE AQUEOUS MEDIUM

In an experiment according to the invention, an aqueous solution of 10 wt. % of glucose, 5 mole % of CrCl3 as catalyst (calculated on glucose) and 45 wt. % choline chloride was combined with THF in a weight/weight ratio of 1:1 and brought to a reaction temperature of 130° C.

In a comparative experiment, 18.8 wt. % of NaCl was used, rather than 45 wt. % of choline chloride (equimolar amount).

The results are presented in Tables 2 and 3 below. Table 2 provides data on the sugar conversion. Table 3 provides data on the selectivity to HMF.

TABLE 2
Sugar conversion [mol glucose reacted/mol glucose fed %]

| | example with 18.8 wt. % | example with 45 wt. % |
|---|---|---|
| | NaCl (comparative) | cholinechloride (invention) |
| t = 40 min | 52% | 63% |
| t = 60 min | 59% | 73% |

TABLE 3
Selectivity towards HMF [mol HMF produced/mol
glucose converted %]]

| | example with 18.8 wt. % | example with 45 wt. % |
|---|---|---|
| | NaCl (comparative) | cholinechloride (invention) |
| t = 40 min | 17% | 47% |
| t = 60 min | 12% | 69% |

From the tables it can be seen that the glucose conversion is higher when cholinechloride is used. Additionally, and even more noticeable, the selectivity for HMF is much higher for the system according to the invention which contains cholinechloride (69% versus 12%). The increased selectivity means that the process according to the invention yields more HMF per gram glucose, and less side products.

### EXAMPLE 3: EXPERIMENT IN CONTINUOUS MODE WITH SUCROSE

In an experiment according to the invention, an aqueous solution of 18 wt. % of sucrose, 10 mole % of CrCl3 as catalyst (calculated on sucrose) and 63 wt. % choline chloride was continuously fed to a stirred tank reactor. At the same time, a continuous flow of THF was also fed to the stirred tank reactor. The ratio of aqueous flow to organic flows was 1:1 wt/wt.

The flows were set in order to achieve 20 minutes residence time. The reaction temperature was achieved by heating in the jacketed stirred tank reactor, and controlled via an oil bath to T=120° C.

The samples are taken after cooling down the mixed outflow to room-temperature and phase separation.

The results are presented in Tables 4 and 5 below. Table 4 provides data on the sugar conversion. Table 5 provides data on the selectivity to HMF and concentrations of HMF in both aqueous and organic phases.

TABLE 4
Sugar conversion [mol sucrose reacted/mol sucrose fed %]

| | Sugar conversion [mol sucrose |
|---|---|
| | reacted/mol sucrose fed %] |
| Average after steady-state | 69 ± 1% |

TABLE 5
Selectivity and HMF extraction

| | Selectivity | | |
|---|---|---|---|
| | towards HMF | | |
| | [mol HMF | % of HMF | % of HMF |
| | produced/mol | in THF | in water |
| | sucrose | phase (calculated | phase (calculated |
| | converted %] | on total HMF) | on total HMF) |
| Average after | 62 ± 1% | 59% | 41% |
| steady-state | | | |

From these tables it can be seen that the process according to the invention allows the conversion of sucrose into HMF through a continuous process with high conversion and high selectivity.

### EXAMPLE 4: EXPERIMENT IN CONTINUOUS MODE WITH GLUCOSE AT DIFFERENT O/A RATIOS

In an experiment according to the invention, an aqueous solution of 10 wt. % of glucose, 5 mole % of CrCl3 as catalyst (calculated on glucose) and 63 wt. % choline chloride was combined with THF in a variable weight/weight ratio and brought to a reaction temperature of 130° C. in a co-current plug-flow reactor. Range of Organic/Aqueous ratio tested comprises: 0.2:1-1:1 wt/wt. The residence time inside the plug-flow reactor was 20 min.

The results are presented in Tables 6 and 7 below. Table 6 provides data on the sugar conversion. Table 7 provides data on the selectivity to HMF.

TABLE 6
Sugar conversion [mol glucose reacted/mol glucose fed %]

| | Sugar conversion [mol glucose |
|---|---|
| | reacted/molglucose fed] |
| example with organic-to- | 75.5% |
| aqueous ratio 0.20:1 | |
| example with organic-to- | 74.2% |
| aqueous ratio 0.49:1 | |
| example with organic-to- | 63.5% |
| aqueous ratio 0.92:1 | |

TABLE 7
Selectivity towards HMF [mol HMF
produced/mol glucose converted %]

| | Selectivity towards HMF [mol HMF |
|---|---|
| | produced/mol glucose converted %] |
| example with organic-to- | 61.1% |
| aqueous ratio 0.20:1 | |
| example with organic-to- | 62.3% |
| aqueous ratio 0.49:1 | |
| example with organic-to- | 71.4% |
| aqueous ratio 0.92:1 | |

From these tables it can be seen that all ranges lead to a good conversion and selectivity. A higher organic-to-aqueous ratio leads to higher selectivity. A lower organic-to-aqueous ratio leads to higher conversion.

### EXAMPLE 5: EXPERIMENT IN CONTINUOUS MODE WITH FRUCTOSE AS STARTING SUGAR

In an experiment according to the invention, an aqueous solution of 10 wt. % of fructose, 5 mole % of CrCl3 as catalyst (calculated on fructose) and 63 wt. % choline chloride was combined with THF in a 0.5 weight/weight ratio and brought to a reaction temperature of 100, 110 or 120° C. in a co-current plug-flow reactor. The residence time inside the plug-flow reactor was 20 min.

The results are presented in Tables 8 and 9 below. Table 8 provides data on the sugar conversion. Table 9 provides data on the selectivity to HMF.

TABLE 8
Sugar conversion [mol fructose reacted/mol fructose fed %]

| | Sugar conversion [mol fructose |
|---|---|
| | reacted/molfructose fed] |
| Temperature 100° C. | 33.7% |
| Temperature 110° C. | 71.1% |
| Temperature 120° C. | 91.9% |

TABLE 9
Selectivity towards HMF [mol HMF
produced/mol sugar converted %]

| | Selectivity towards HMF [mol HMF |
|---|---|
| | produced/mol fructose converted %] |
| Temperature 100° C. | 61.0% |
| Temperature 110° C. | 66.7% |
| Temperature 120° C. | 68.5% |

From the tables it can be seen that higher temperatures lead to higher conversion and a benefit in terms of selectivity.

1. A process for producing 5-hydroxymethylfurfural (HMF) comprising a) a step of converting a carbohydrate into HMF, the converting step comprising providing a reaction medium comprising carbohydrate, catalyst, water, tetrahydrofuran (THF), and salt to form a biphasic solvent system comprising an aqueous phase and a THF phase and
b) a step of separating the THF phase and the aqueous phase, to provide a separate THF phase and a separate aqueous phase,
wherein an organic quaternary ammonium salt is present.

2. The process according to claim 1, wherein the carbohydrate is selected from the group of lignin, sugars, starches, celluloses, and gums.

3. The process according to claim 2, wherein the carbohydrate is a sugar selected from C5 sugars such as arabinose, xylose and ribose; C6 sugars such as glucose, fructose, galactose, rhamnose and mannose; and C12 sugars such as sucrose, maltose and isomaltose.

4. The process according to claim 1, wherein the organic quaternary ammonium salt is an organic quaternary ammonium chloride.

5. The process according to claim 1, wherein the catalyst is selected from the group of halides of chromium and aluminium.

6. The process according to claim 1, wherein the organic quaternary ammonium salt is present in an amount of at least 10 wt. %, calculated on the total of carbohydrate, water, and salt.

7. The process according to claim 1, wherein the weight ratio of THF to aqueous solution containing carbohydrate and salt is in the range of 0.05:1 to 10:1.

8. The process according to claim 1, wherein the reaction is carried out at a temperature in the range of 80-180° C. for a period of 1 minute to 4 hours.

9. The process according to claim 1, wherein the step of separating the THF phase and the aqueous phase to provide a separate THF phase and a separate aqueous phase is carried out at a temperature at or below the reaction temperature.

10. The process according to claim 1, wherein the separated THF phase which contains HMF is subjected to a separation step, with THF being separated off, optionally after addition of water to the HMF-containing THF phase.

11. The process according to claim 1, wherein one or more of the following steps take place:
THF resulting from the separation of HMF from the HMF-containing THF phase is recycled to the reaction step,
aqueous phase comprising organic quaternary ammonium salt and, where the catalyst is homogeneous, catalyst is recycled to the reaction step, optionally after intermediate purification and/or concentration.

12. The process according to claim 1, which comprises the further step of converting the HMF to FDCA.

13. The process according to claim 12, which comprises the further step of reacting the FDCA in a polycondensation reaction with ethyleneglycol to form poly(ethylenefurandicarboxylate).
